(19) United States
(12) Patent Application Publication
(10) Pub. No.: US 2003/0003114 A1
(43) Pub. Date: Jan. 2, 2003

(54) Enzyme-based anti-cancer compositions and methods

(76) Inventors: Xing Qing Pan, Columbus, OH (US); Robert Lee, Columbus, OH (US)

Correspondence Address: CALFEE HALTER & GRISWOLD, LLP, 800 SUPERIOR AVENUE, SUITE 1400, CLEVELAND, OH, 44114, US

(21) Appl. No.: 10/104,475

(22) Filed: Mar. 22, 2002

Related U.S. Application Data
(60) Provisional application No. 60/278,026, filed on Mar. 22, 2001.

(51) Int. Cl. A61K 9/00

(52) U.S. Cl. 424/400

(57) ABSTRACT

A composition for treating cancer is provided. The composition is a hexosaminidase covalently attached to a cancer cell targeting ligand and improves selectively of the hexosaminidase for tumor cells. In certain embodiments, the hexosaminidase is alternately chitinase (N-acetyl-glucosaminohydrolase), chitosanase, or N-acetyl-hexosaminidase and the targeting ligand is either a monoclonal antibody, an antibody fragment immunospecific to a tumor cell or cancer cell antigen, epidermal growth factor (EGF), fibroblast growth factor (FGF), transferrin, or folic acid. Also provided is a method for treating cancerous tumors comprising administering the composition to a patient that has a cancerous tumor.

This application claims priority from U.S. Provisional Patent Application Serial No. 60/278,026, which was filed on Mar. 22, 20001.

This invention was made, at least in part, with government support under NIH grant No. ROI CA79758-O1A1. The U.S. government has certain rights in this invention.

### FIELD OF THE INVENTION

The present invention relates generally to the treatment of cancerous tumors with enzyme-based compositions, and specifically to the treatment of cancerous cells and tumors with hexosaminidases.

### BACKGROUND OF INVENTION

Tumorigenesis is typically accompanied by marked changes in the patterns of gene expression and post-translational modifications of gene products. These changes can lead to highly distinctive cellular phenotypes and membrane compositions among tumor cells. For example, polycarbohydrate structures and their organization on the surface of neoplastic cells can be different from those of normal cells. Common changes in cell surface carbohydrates in tumor cells include the appearance of high molecular weight glycoproteins that are not found in normal cells. Tumor-specific glycolipids may also be present. The changes in carbohydrate composition can be more pronounced in tissues of metastatic lesions. Oligosaccharides on these glycoproteins, and possibly glycolipids, can play an important role in determining the biological behavior of the tumor.

Changes in the tumor cell membrane are recognized by the immune system and distinctive carbohydrates have been identified as tumor-associated antigens. Tumor-specific

glycoproteins and glycolipids have been evaluated as potential vaccines for tumor immunotherapy in clinical trials. The difference in surface carbohydrate composition between the tumor cells and normal cells, therefore, presents an excellent opportunity in the search for tumor treatment strategies that selectively attack tumor cells. Although much effort has been devoted to this approach, the results and data generated thus far have been inconclusive.

Existing chemotherapy agents, such as doxorubicin, taxol, and cis-platin; and biological response modifiers such as tumor necrosis factors, interferon, and interleukin-2, have limited demonstrable effectiveness against cancer. Initial response of the cancer to a particular anti-cancer agent is often followed by the development of resistant tumors which exhibit multi-drug resistance due to up-regulation of the P-glycoprotein. Additionally, these agents often exhibit dose-limiting toxicity. For example, doxorubicin causes both acute and cumulative cardiotoxicity. Also, treatment is often associated with severe side effects, such as myelotoxicity and suppression of the immune system, which leaves the patient vulnerable to opportunistic infections and the development of new cancer.

Accordingly, it is desirable to have additional methods and compositions for treating cancers.

### SUMMARY OF THE INVENTION

In accordance with the present invention, it has been discovered that hexosaminidases can be used to inhibit growth of cancer cells and to treat cancerous tumors and neoplasias.

Thus, the present invention provides, as a new composition of matter, a hexosamindase that is covalently attached to a targeting ligand (hexosaminidase-ligand conjugate). The hexosaminidase has, as its substrate, glycoproteins or glycolipids that are present on the surface of a tumor cell. Such glycoproteins or glycolipids comprise acetylated or unacetylated hexosamines. Such hexomamines may be glucosamines. Preferably, the hexosaminidase of the composition is a glucosaminidase or N-acetyl glucosaminidase. The targeting ligand of the composition binds or associates with molecules or structures present on the surface of tumor cells, thus bringing the attached hexosaminidase into close proximity to the tumor cell. In one embodiment, the hexosaminidase is directly attached to the targeting molecule. In another embodiment, the hexosaminidase is indirectly attached to the targeting molecule through the use of a linker molecule, for example. In one aspect, the linker molecule is polyethylene glycol (PEG).

The present invention also provides methods for treating cancer in a patient. The methods comprise administering a pharmaceutical composition comprising a hexosaminidase to a patient with a cancerous tumor or neoplasia. In one embodiment, the pharmaceutical composition comprises one or more hexosaminidases and is injected into the tumor. In another embodiment, the pharmaceutical composition comprises a hexosaminidase covalently attached to a selected targeting ligand. The hexosaminidase may be directly attached, or indirectly attached to the targeting molecule, through the use of a linker molecule. The pharmaceutical composition comprising the hexosaminidase-ligand conjugate is administered to the patient intraperitoneally, intravenously, intratumorally, or by other conventional methods.

The present invention also provides methods for treating cancer in a patient that comprise administering a chitin derivative to a patient to stimulate chitotriosidase by macrophages, monocytes or other cells in the patient.

### BRIEF DESCRIPTION OF THE FIGURES

The present invention may be more readily understood by reference to the following drawings wherein:

FIG. 1 is a graph showing survival of cultured cells in the presence of various concentrations of chitinase;

FIG. 2 is a graph of tumor size in SCID mice after intratumoral injection of chitinase;

FIG. 3 shows photographs of human tumors and cells in SCID mice treated with chitinase;

FIG. 4 shows electron micrographs of human tumor cells cultured in the presence and absence of chitinase; and

FIG. 5 is a graph of growth of 24 JK murine sarcoma cells in C57BL/6 mice after IP injection of chitinase.

### DETAILED DESCRIPTION OF THE INVENTION

### Definitions

Unless otherwise indicated, the following terms used in this document have the following meanings:

“Polycarbohydrate” refers to a polymer comprised of repeating saccharide units. Herein, polycarbohydrate includes polymers of saccharides that also contain hexosamines.

“Targeting ligand” refers to a molecule with affinity for a molecule located on the surface of a tumor cell. Preferably, a molecule located on the surface of the tumor cell is specific to the tumor or present at levels higher than those present in non-tumor cells.

“Hexosaminidase-ligand conjugate” refers to a molecule comprised of a hexosaminidase covalently attached to a targeting ligand. Such hexosaminidase-ligand conjugates may or may not include a linker molecule.

“Linker” refers to a molecule that serves to attach a hexosaminidase to a targeting ligand.

“Tumor” refers to a spontaneous, new growth of tissue in the body that forms an abnormal mass. Tumors are comprised of cells and such cells are known as tumor cells. Tumors and cells derived from tumors can be either benign or malignant (see below). “Neoplasm” is essentially synonymous with tumor.

“Units” refers to a measurement of activity of an enzyme and is normally described in terms of the amount of enzyme that reacts upon a certain amount of substrate in a given time or amount of enzyme that produces a certain amount of reaction product in a given time. Herein, for chitinase, units is defined as the amount of enzyme that liberates 1.0 mg of N-acetyl-D-glucosamine from chitin per hour at pH 6.0 at 25° C. in a 2 hour assay.

“Cancer” refers to a malignant tumor or neoplasm. Cells that are malignant have a variety of properties that benign cells and non-tumor cells do not have. Malignant cells invade, grow and destroy adjacent tissue, metastasize, and usually grow more rapidly than benign tumor cells.

“Chitin derivative” refers to molecules that can stimulate chitinase production by macrophages or monocytes. Chitin derivatives include parts of chitin molecules and chitin molecules that are chemically modified, as long as such molecules stimulate chitinase production.

“Fusion protein” refers herein to a protein resulting from expression of a fusion gene. The fusion gene comprises a single open reading frame which encodes open reading frames from two or more distinct proteins which are directly linked by a peptide bond or through a linker linking unit comprising one or more amino acids. The fusion proteins referred to herein comprise a hexosaminidase and a tumor cell targeting ligand.

“Expression vector” refers to one or more DNA coding sequences additionally containing adjacent or surrounding DNA sequences needed for expression of the gene or genes. Expression vectors can be plasmids, viruses, or other DNAs that are well known in the art. Expression of a gene refers to transcription of the gene into mRNA, and translation of the mRNA into protein, and optional additional processing of the protein.

In accordance with the present invention, it has been found that growth and survival of tumor and neoplastic cells are particularly sensitive to hexosaminidases. Hexosaminidases are enzymes that have as their substrate a polycarbohydrate comprising one or more hexosamines. The polycarbohydrate molecules that are attached to proteins or lipids on the surface of cells, forming glycoproteins or glycolipids, respectively. It has been found that hexosaminidases inhibit growth and kill transformed cells in culture while nontransformed cells are relatively resistant to the growth inhibition and killing effects. Breast, lung, colon and prostate carcinomas are effectively growth inhibited and killed by injection of hexosaminidases directly into the tumors in animals. Hexosaminidases can also be targeted to particular cancer cells in an animal by covalently attaching a targeting ligand to a hexosaminidase. Such a molecule is called a “hexosaminidase-ligand conjugate.” The conjugate may or may not additionally contain a linker molecule, polyethylene glycol for example, that serves to attach the hexosaminidase to the targeting ligand. When administered to a patient, the targeting ligand of the hexosaminidase-ligand conjugate binds to the molecule or structure for which it has affinity on the surface of cells comprising a tumor. This binding brings the hexosaminidase of the conjugate into close proximity with the polycarbohydrate substrates on the tumor cell surface, allowing the enzyme to function. The present invention also includes methods for stimulating production of natural hexosaminidases in a patient by administration of chitin derivatives.

In a preferred embodiment of the present invention, chitinase exhibits remarkable antitumor activity without significant systemic toxicity. In the preferred embodiment, chitinase is utilized as a therapeutic agent for the treatment of a variety of cancerous tumors. Even high chitinase levels do not appear to cause toxicity in normal tissues.

### Cell Surface Carbohydrates

Polycarbohydrates are found on the surface of mammalian cells, particularly on the surface of cancer cells. On the cell surface, the polycarbohydrates are part of part of proteins (i.e., glycoproteins) or lipids (i.e., glycolipids).

Polycarbohydrates are polymers of saccharides. A simple saccharide is a monosaccharide. The monosaccharides of interest in the present invention have the chemical formula (CH₂O)₆, and are called hexoses. The hexoses can be of a number of different types which include glucose, galactose, mannose, and others. Often, the hexose in the cell surface polycarbohydrates of present interest are glucoses. The hexoses can be modified, for example by replacement of an alcohol group (—OH) on one or more carbon atoms of the sugar backbone with an amine group (—NH₂). One such replacement occurs at carbon 2 of the hexose backbone. When the replacement occurs in a glucose molecule, the molecule is called 2-amino-2-deoxy-D-glucose or D-glucosamine. Glucosamine is commonly found in polycarbohydrates of natural origin. One such polycarbohydrate is chitosan. When the alcohol group at carbon 2 of a glucose molecule is replaced with an N-acetyl group, the molecule is called N-acetyl-D-glucosamine, which is found in chitin.

Polycarbohydrates are formed from polymerization of any of the monosaccharides described above, or of combinations of more than one monosaccharide, called disaccharides, trisaccharides, and the like. The polymerization occurs by formation of glycosidic bonds between two saccharides. Glycosidic bonds form between the anomeric hydroxyl group of a saccharide and the hydroxyl of a second saccharide. The molecule formed from such a reaction is called a glycoside.

The polycarbohydrates of interest in the present invention are those containing hexosamines. The hexosamines may be acetylated or unacetylated. Preferably, the acetylated or unacetylated hexosamine is a glucosamine. Chitin is one polycarbohydrate that contains glucosamine. Chitin is the primary component of the exoskeleton in a large number of organisms, including the cell walls of fungi and of some algae and the shells or cuticles of arthropods. Although mammalian cells contain no chitin, certain carbohydrates distinctively expressed on the surface of cancer cells contain glucosamine derivatives, and therefore may be susceptible to chitinase-catalyzed hydrolysis.

### Hexosaminidases

Glycoside hydrolases or glycosidases are enzymes which hydrolyze glycosidic bonds of various saccharides. Glycosidases may include any enzyme for which a polycarbohydrate, such as chitin, is a substrate. Preferably, the glycosidases of the present invention are hexosaminidases that cleave polycarbohydrates comprising glucosamine, N-acetyl glucosamine or poly-N-acetyl-D-glucosamine (i.e., glucosaminidases), and include chitinases (EC 3.2.1.14, family 18,19), chitosanases (EC 3.2.1.132, family 46), or N-acetyl-hexosaminidases (EC 3.2.1.52, family 3 & 20). Chitinases are also referred to as endochitinases, chitotriosidase in humans, chitodextrinase, endo-beta-N-acetylhexosaminidase, and poly-beta-glucosaminidase. N-acetyl-hexosaminidases include chitobioses (EC 3.2.1.30, family 20) and exochitinases. The hexosaminidases of the present invention include both endoglycosidases which catalyze the internal cleavage of the carbohydrate chain, and exoglycosidases which catalyze the cleavage of terminal glucosamine residues from the carbohydrate chain.

One example of a suitable hexosaminidase is the enzyme chitinase. Chitinase is a carbohydrate hydrolase. Its natural substrate is chitin, which consists of a group of polycarbohydrates rich in N-acetyl-D-glucosamines. Chitinase catalyzes the hydrolysis of β-1,4 linkages of N-acetyl-D-glucosamine polymers of chitin. Commercially available chitinases are isolated from Serratia marcescens (Sigma C7809), Vibrio parahemolyticus, or Streptomyces griseus (Sigma C1525). Chitotriosidase (Sigma C9830), an enzyme exhibiting structural similarity to chitinase, is produced in certain human lymphocytes including activated macrophages. The normal substrate for chitotriosidase is poly-β-D-glucosamines. The cDNA gene for chitotriosidase has been cloned and sequenced, and the associated protein has been purified and partially characterized. β-N-Acetylhexosaminidase (Sigma A7708) is another related enzyme whose normal substrate is N-Acetyl-β-D-hexosamide.

The hexosaminidases of the present invention can be derived from any source and include hexosaminidases that come from bacteria, yeast, fungus, plants or mammalian sources.

### Testing for Hexosaminidases that Have Anti-Cancer Activity

Hexosaminidases that have anti-cancer cell activity are identified by a variety of methods using cultured cells and animals. Good results have been obtained by testing the effect of hexosaminidases on the viability of cultured normal and tumor cells is tested by culturing normal cells (e.g., spleen cells from a mouse) and tumor cells (e.g., human oral carcinoma KB cells) in vitro and then adding various concentrations of the hexosaminidase to the cells. Different concentrations of the hexosaminidases are used. At various times after addition of the hexosaminidase, the viability of the cells is determined using one of a variety of methods. One method is the MTT assay. In this assay, cells are exposed to MTT, 3-(4,5-dimethylthiazol-2-yl)2,5-diphenyltetrazolium bromide, which is taken into the cells and reduced by mitochondrial dehydrogenase to a purple formazan, a large molecule which is unable to pass through intact cell membranes, and therefore accumulates in healthy cells. The ability of cells to reduce MTT is an indication of mitochondrial integrity and activity, which is interpreted as a measure of viability.

The effect of hexosaminidases on survival of tumor cells is also tested by administering hexosaminidases to experimental animals that have various tumors. Preferably, the tumors are human tumors or cancer xenograft tissues and the experimental animal is a SCID mouse. Good results have been obtained by transplanting tumor cells or tissues subcutaneously in the animal or behind the fat pad of the mouse. After the tumor cells or tissues are transplanted into the animal, the tumors cells grow and divide. When the tumors reach a visible size (e.g., 0.5-0.8 cm³) in the SCID mice, a hexosaminidase is dissolved in PBS and various amounts of the hexosaminidase are injected into the tumor (i.e., intratumor injection), injected intraperitoneally (IP) into the animal, or intravenously (IV) into the animal. Single or multiple injections are used. The size of the tumor is subsequently measured to determine the effect of the enzyme on tumor growth and survival. A decrease in size of the tumors is indicative of anti-tumor activity of the hexosaminidase. Preferably, administration of the hexosaminidase results in a significant size reduction of the tumor. More preferably, the hexosaminidase eliminates the tumor from the SCID mouse. Preferably, such animals will remain free from tumors in the future. In the cases in which a hexosaminidase does not possess anti-tumor activity, the tumors show progressive growth and the animals eventually die or have to be removed from the study due to excessive tumor burden, generally between 20 to 60 days from the time of implantation, depending of the tumor.

In the case where a hexosaminidase is tested for anti-tumor activity on murine tumor cells or tissues, a syngeneic tumor model is used. In this model, tumor cells or tissues are implanted in C57BL/6 mice. As in the SCID mouse model described above, the implanted tumors are allowed to grow to a size of approximately 50 to 100 mm³ before various amounts of the hexosaminidase are administered to the animal, by any of the means described above. The decrease in tumor size or, preferably, elimination of the tumor from the mouse, is subsequently observed.

In both the SCID and C57BL/6 models described above, the anti-tumor activity of a hexosaminidase is compared to the anti-tumor activity of a known anti-tumor agent. For example, mice containing tumors are treated with doxorubicin. A suitable dose of doxorubicin is 5 mg per kg of body weight of the mice.

In both the SCID and C57BL/6 models described above, mice that are administered a hexosaminidase are observed for visible signs of systemic toxicity. Such toxicity is compared with toxicity that is present in control animals, animals administered doxorubicin for example. Generally, hexosaminidase enzymes exhibit low systemic toxicity and, in addition, do not induce drug resistance, are effective against very large tumors (up to about 1 gram in mice), are effective against tumors of many different types, and do not exhibit reoccurrence following cure.

### Targeting Ligands and Linkers

The present invention provides compositions for treating cancerous tumors and neoplasias. The composition comprises a hexosaminidase and a targeting ligand covalently bound to the hexosaminidase. Such a composition is called a hexosaminidase-ligand conjugate. In such a composition, the hexosaminidase is covalently bound to the targeting ligand. Preferably, the hexosaminidase is a glucosaminidase. Examples of suitable hexosaminidases are chitinase (N-acetyl-glucosaminohydrolase), chitosanase, or N-acetyl-hexosaminidase.

The targeting ligand that is part of the composition improves tumor cell selectivity of the composition. The targeting ligand binds or associates with molecules or structures present on the surface of tumor cells, thus bringing the attached hexosaminidase into close proximity to the tumor cell, allowing the hexosaminidase to contact its substrate on the tumor cell surface. The particular tumor to which the hexosaminidase-ligand conjugate associates is controlled by suitable selection of the targeting ligand that is to be part of the composition. Preferably, a targeting ligand is selected to bind to a molecule or structure on the surface of the tumor to be treated. Preferably, a molecule or structure to which the targeting ligand binds is not present on cells other than the cells of the tumor to be treated. A targeting ligand may also be selected to bind to a molecule or structure that is present both on tumor cells and on non-tumor cells. In this case, however, it is preferable that the molecule or structure is present in greater amounts on the tumor cell than on the non-tumor cell. Preferably, the molecule or structure is present at least at 10-fold higher levels on tumor cells than non-tumor cells. Such molecules or structures may be present at 1000-fold or even higher levels on tumor cells as compared to non-tumor cells. Targeting ligands can be chosen to make hexosaminidase-ligand conjugates that are effective against different tumors.

Examples of suitable targeting ligands are a monoclonal antibody or an antibody fragment immunospecific to a tumor cell or cancer cell antigen. The (HER2 antibody) is one example. There are a variety of other antigens that are present on tumor cells but not on non-tumor cells. Antibodies directed against any of these tumor-specific antigens are possible. Other possible targeting ligands include growth factors, such as epidermal growth factor (EGF), insulin and fibroblast growth factor (FGF), are suitable targeting ligands. Cytokines (e.g., interleukin-2) are suitable targeting ligands. Other suitable targeting ligands include transferrin, folic acid and somatostatin. There are a variety of other targeting ligands that can be used. Any molecule, compound or substance that can bind or associate with a cell can be used. A preferable characteristic of such a targeting ligand is that is bind to the desired tumor cells but not bind or minimally bind to other cells, such that specificity of the hexosaminidase-ligand conjugate for the tumor cells is achieved.

In the preferred embodiment, the targeting ligand is folate or folic acid. Folate is a vitamin with a molecular weight of 441.4. Folate retains affinity for the folate receptor upon derivatization by means of its gamma-carboxyl to a wide variety of molecules. Folic acid has many unique advantages as a tumor targeting ligand compared with monoclonal antibodies, including (i) rapid tissue distribution and clearance, resulting in high tumor to background tissue ratios, (ii) convenient availability, (iii) defined conjugation chemistry, and (iv) non-immunogenicity.

The folate receptor is overexpressed in many human tumors including over 90% of ovarian carcinomas. Folate has been used for targeting protein toxins to cultured tumor cells. Folate conjugates have been shown to be taken into tumor cells by means of folate receptor-mediated endocytosis.

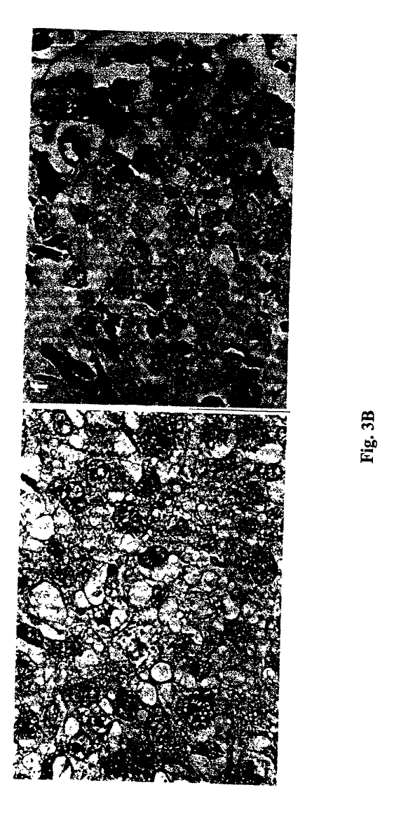

Although folate receptor is not found in prostate cancer, the prostate-specific membrane antigen (PSMA) has been found to bind folate derivatives with high affinity. Folate-conjugation, therefore, presents a method for targeting of diagnostic and therapeutic agents to prostate cancer cells. FIG. 3 illustrates the mechanism of folate-mediated drug targeting to prostate cancer cells.

PSMA is a type II multi-spanning membrane protein with a molecular weight of 100 kDa. Mostly undetectable in normal tissues, PSMA is consistently overexpressed in prostate carcinomas. PSMA is also found to be overexpressed in endothelial cells of a wide spectrum of malignant neoplasms but not in normal vascular endothelium of non-cancerous tissues.

In one embodiment of the hexosaminidase-ligand conjugate, the hexosaminidase is directly attached to the targeting ligand. Direct attachment generally means that no molecules or substances other than the hexosaminidase and targeting ligand are part of the composition. In another embodiment of the hexosaminidase-ligand conjugate, the hexosaminidase and targeting ligand are indirectly attached to one another, through the usage of another molecule, for example. The molecule or substance that serves to attach the hexosaminidase indirectly to the targeting molecule is called a “linker.” The linker is preferably stable, and may be acid sensitive, reducible (containing a disulfide bond) or protease sensitive. Linkers can also be peptides.

The attachment of the linker to both the hexosaminidase and the targeting ligand is through covalent bonds. One such linker molecule is polyethylene glycol (PEG) or other flexible hydrophilic polymers. There can be a variety of reasons for choosing to use a linker molecule in a hexosaminidase-ligand conjugate. One such reason is that the linker molecule may facilitate or make possible the attachment of the hexosaminidase to the targeting ligand. The linker molecule may facilitate association of the targeting ligand portion of the hexosaminidase-ligand conjugate with a molecule or substance on the surface of the tumor cell. The use of PEG as a linker, for example, presumably reduces the immunogenicity of the hexosaminidase portion of the composition and protects the hexosaminidases from degradation by proteases, thus improving the potential pharmokinetic and therapeutic effects of the composition.

The hexosaminidase-ligand conjugate, which may or may not include a linker, encompasses variants, especially genetic variants, of the hexosaminidase and targeting ligand. These variants include truncated or mutated hexosaminidase or targeting ligand.

### Making Hexosaminidase-Ligand Conjugates

The separate components of the hexosaminidase-ligand conjugate (i.e., hexosaminidase, targeting ligand, and optional linker) are attached to one another through the use of covalent chemical bonds. Once a specific hexosaminidase, targeting ligand, and optional linker are chosen, there are a variety of ways by which these components can be covalently attached to one another. Preferably, such covalent attachment results in a hexosaminidase-ligand conjugate in which the individual components are functional, meaning that the hexosaminidase is able to hydrolyze a particular glycosidic bond, and the targeting ligand is able to bind or associate with a specific cell.

It is well known in the art that reactive chemical groups react with one another to form covalent bonds. In the present invention, at least one chemically reactive groups is present on each component of the hexosaminidase-ligand conjugate that is to be attached to one another. For example, in order to make a composition that consists of a hexosaminidase and a targeting ligand, there is at least one chemically reactive group on the hexosaminidase and one chemical group on the targeting ligand. It is not a requirement that the reactive groups on the separate components are identical to one another. However, whatever the identity of the reactive groups on the separate components, they are reactive with one another, such that a covalent bond is formed. There are a variety of reactive chemical groups that are known in the art and can be used to form the covalent bonds that attach the components of the hexosaminidase-ligand conjugate to one another. Some examples of chemically reactive groups are —NH₂, —COOH, —SH, —CHO and —SO₄ groups. The covalent bonds formed may be amide, ester, ether, thioester, isourea, Schiff's base, or hydrazone bonds.

If the targeting ligand that is selected is a protein, recombinant DNA techniques can be used to create a fusion of the genes encoding the targeting ligand and the hexosaminidase. The fusion gene is expressed in a suitable host, a bacterium for example. The expressed fusion protein is purified from the host and used as the hexosaminidase-ligand conjugate of the present invention.

### Pharmaceutical Compositions

The hexosamindases and hexosaminidase-ligand conjugates are preferably part of a pharmaceutical composition that is intended for administration to a patient. The particular pharmaceutical composition will depend on the method by which the composition is administered to a patient. However, pharmaceutical compositions routinely comprise salt, buffering agents, preservatives, adjuvants, other vehicles and, optionally, other therapeutic agents in addition to the hexosaminidases or hexosaminidase conjugates.

The pharmaceutical compositions, generally speaking, may be administered using any mode that is medically acceptable, meaning any mode that produces the desired anti-tumor activity without causing clinically unacceptable adverse effects. Such modes of administration include parenteral routes (e.g., intravenous, intra-arterial, subcutaneous, intramuscular, mucosal or infusion), but may also include oral, rectal, topical, nasal or intradermal routes. Other delivery systems can include time-release, delayed release or sustained release delivery systems. Such systems can avoid repeated administrations, increasing convenience to the subject and the physician. Many types of release delivery systems are available and known to those of ordinary skill in the art.

Compositions suitable for parenteral administration are preferred and conveniently comprise a sterile aqueous or oleaginous preparation of hexosaminidase or hexosaminidase conjugate, which is preferably isotonic with the blood of the recipient. This aqueous preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa. The pharmaceutical compositions may conveniently be presented in unit dosage form and may be prepared by any of the methods well-known in the art of pharmacy.

### Method of Treatment

The present invention also provides methods of treating a cancer in a subject. In one embodiment, the method of treatment is administration of one or more hexosaminidases to the subject. The specific hexosaminidases may be chitinase, chitosanase, N-acetyl-hexosaminidase, or any of the other enzymes described herein. The hexosaminidases are administered to the subject by a variety of methods. These routes of administration include injection of the hexosaminidase directly into the tumor (i.e., intratumoral administration). Other methods include injection of the hexosaminidase intravenously, intraperitoneally, intramuscularly subcutaneously or intracerebrally. Other methods of administering the hexosaminidase can be used.

In another embodiment, the method of cancer treatment is by administration of one or more hexosaminidase-ligand conjugates, as described above, to the subject. The routes of administering the conjugate include all methods described above for administration of the hexosaminidase.

The above methods for cancer treatment (i.e., administration of a hexosaminidase and administration of a hexosaminidase-ligand conjugate) may be combined together and/or may be combined with other known methods for treating a particular cancer. Such methods may include chemotherapy, surgery, radiotherapy, photodynamic therapy, gene therapy, antisense therapy, enzyme prodrug therapy, immunotherapy, fusion toxin therapy, antiangiogenic therapy, or any combination of these therapies. In this embodiment, preferably, the hexosaminidase is either chitinase, chitosanase, or N-acetyl-hexosaminidase, and the targeting ligand is alternately a monoclonal antibody or antibody fragment immunospecific to a tumor cell or cancer cell antigen, epidermal growth factor (EGF), fibroblast growth factor (FGF), transferrin, folic acid, or any other molecule that selectively binds to a tumor or cancer cell.

In the administration of both hexosamindases and hexosaminidase-ligand conjugates, described above, drug delivery devices such as infusion pumps may be utilized, or the composition may be administered in the form a denatured pellet, or in hydrogel, or nano or microparticles.

### Dosage of Hexosaminidase or Hexosaminidase-Ligand Conjugate

The hexosaminidases and hexosaminidase-ligand conjugates of the present invention can be administered to humans in an amount that inhibits growth of a tumor or, preferably, eliminates the tumor from the body. The amount of hexosaminidase or hexosaminidase-ligand conjugate that eliminates a tumor will vary with the particular tumor being treated, the age and physical condition of the subject being treated, the severity of the condition, the duration of the treatment, the nature of the concurrent therapy (if any), the specific route of administration and like factors within the knowledge and expertise of the health practitioner. In the event that a response in a subject is insufficient at the initial doses applied, higher doses (or effectively higher doses by a different, more localized delivery route) may be employed to the extent that patient tolerance permits. Multiple doses are contemplated to achieve good results.

### Stimulating Production of Natural Hexosaminidases

Another method of the present invention for treating cancerous tumors includes administering a chitin derivative to a subject to stimulate chitinase production by human macrophages or monocytes. Chitinase is naturally produced in human macrophages and destroys the cell wall of invading bacteria and yeast in the event of an infection. Chitinase production is stimulated by a chitin derivative. If sufficient chitinase can be produced by the body to effect anti-tumor activity, administration of chitin derivatives may provide an alternative method for prostate cancer treatment.

In this embodiment, the chitin derivatives include chitosan and derivatives of chitosan. Alternatively, chitinase may be cloned into a gene vector and be administered as a gene therapy. This can be done by administering the gene vector to the subject such that the vector expresses its genes in cells of the subject. This can also be done by expressing the gene vector in mammalian cells in vitro, then administering the mammalian cells to the subject.

Compared to chitinase protein administration, chitin derivatives have many advantages, including non-immunogenicity and favorable pharmacokinetic properties. The present invention includes a series of chitin derivatives with potential chitinase stimulatory activities.

### EXAMPLES

The invention may be better understood by reference to the following examples, which serve to illustrate but not to limit the present invention.

### Example 1

### Survival of Cultured Normal and Tumor Cells in Media Containing Chitinase

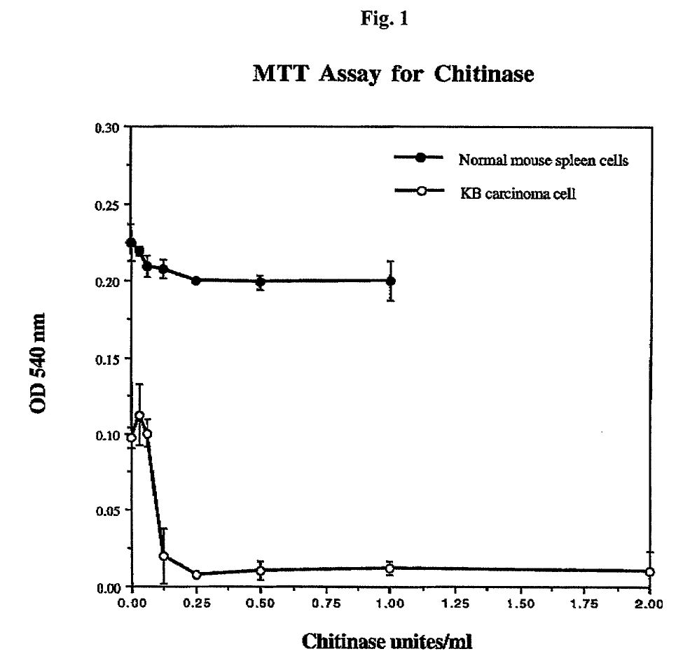

Fifty thousand spleen cells from a normal mouse, or human oral carcinoma KB cells were cultured. Chitinase (from Streptomyces griseus) was dissolved in PBS and added to the cells at the indicated concentrations shown in FIG. 1 and the cells were cultured at 37° C. Three days later, MTT (3-(4,5-dimethylthiazol-2-yl)-2,5-diphenyltetrazolium bromide) dye reduction assays were performed to determine the viability of the cells in the presence of the chitinase. MTT is a chemical compound, which is converted to a blue formazan product in living cells. This product was dissolved in a solvent (95% ethanol:DMSO; 1:1), added to the cells and the absorbance (540 nm) was determined. The absorbance of the formazan is linearly proportional to the number of viable cells in the culture. The data in FIG. 1 show that chitinase efficiently kills the tumor cells (KB carcinoma cells) at concentrations of between 0.025 to 0.05 units of chitinase per ml of culture medium. Concentrations of chitinase up to 1.0 units per ml of culture medium had little effect on the viability of the normal mouse spleen cells.

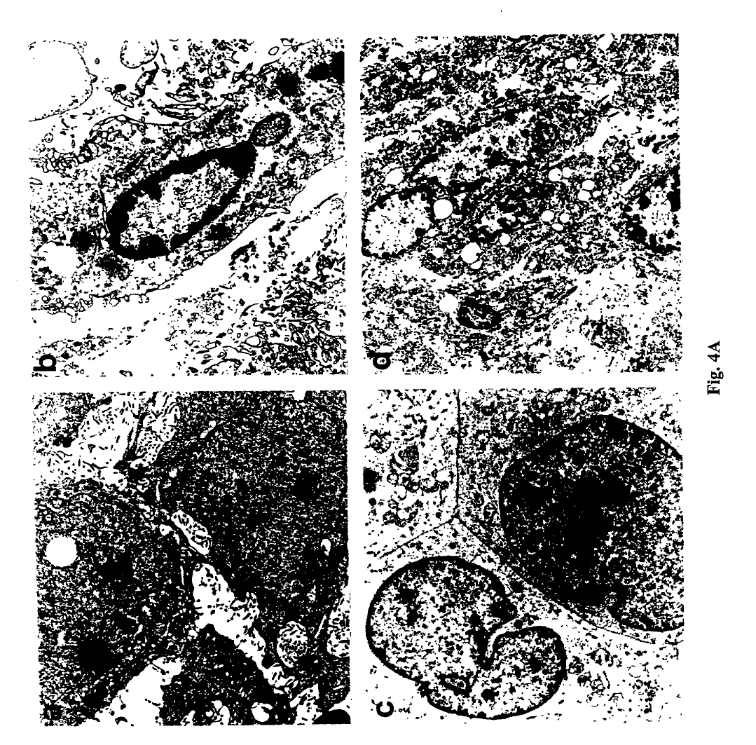

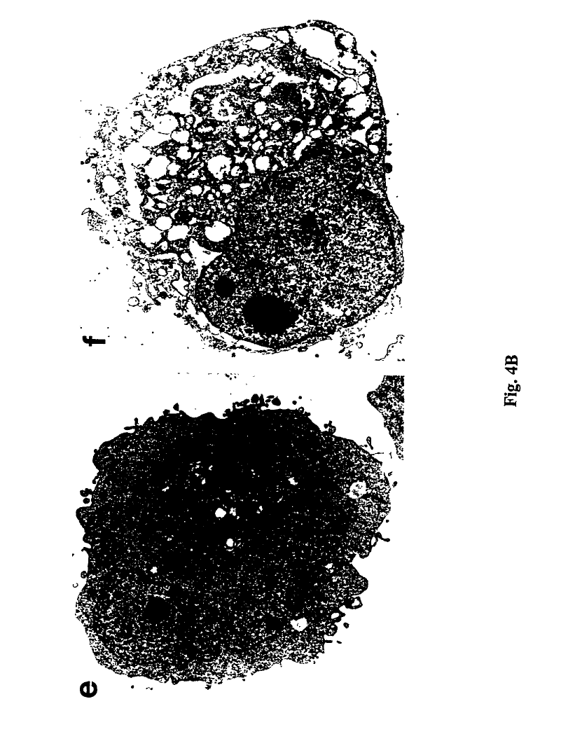

In other studies, cultured lung cancer, melanoma, prostate cancer LNCaP cells and breast cancer MCF-7 cells were treated with bacterial chitinase resulting in observance of significant cellular damage within hours of chitinase treatment and ultimately leading to cell death. FIG. 4 shows electron micrographs of lung cancer cells (FIG. 4a and b), melanoma cells (FIG. 4c and d), and MCF-7 breast cancer cells (FIG. 4e and f), cultured in the absence of chitinase (FIG. 4a, c and e) and after 7 hours of culture in medium containing 0.5 units of chitinase per ml of medium.

### Example 2

### Survival of SCID Mice Carrying Human Colon Cancer Xenografts After Chitinase Injection

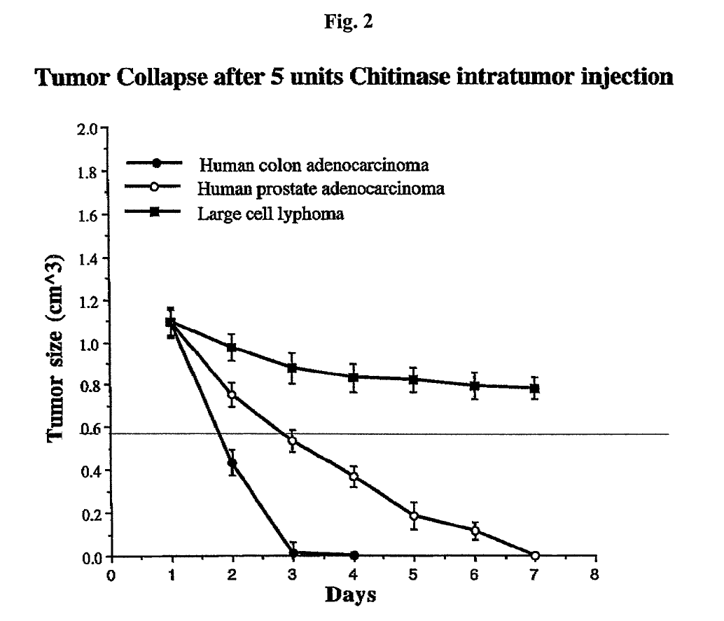

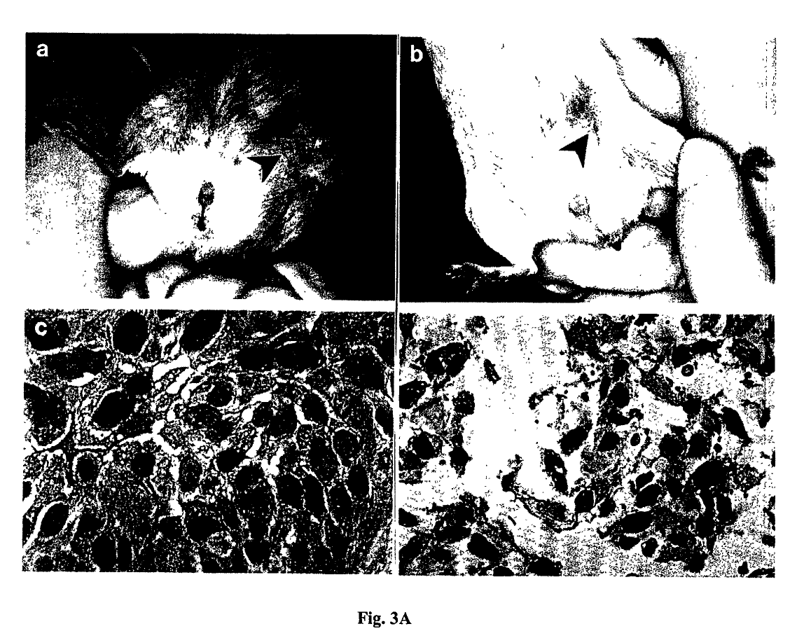

Moderately differentiated adenocarcinoma from human colon was obtained from patients as a biopsy sample and a 0.1 cm³ piece of the biopsy was implanted into SCID mice. After the tumors grew to a size of between 0.3-0.6 cm³, 5 units of chitinase was injected into the tumor (day 1). Subsequently, the size of the tumors was measured daily. The results (FIG. 2) showed that human colon cancer was effectively eliminated from the animals by the chitinase over a period of 2 days.

### Example 3

### Survival of SCID Mice Carrying Human Lung Cancer Xenografts After Chitinase Injection

Moderately differentiated squamous cell carcinoma from human lung was obtained from patients as a biopsy sample and a 0.1 cm³ piece of the biopsy was implanted into SCID mice. After the tumors grew to a size of between 0.3-0.6 cm³, 5 units of chitinase was injected into the tumor (day 1). Subsequently, the size of the tumors was measured daily. The results showed that the lung tumors were as effectively eliminated from the mouse by chitinase as was the human colon cancer as described in Example 2.

FIG. 3 shows photographs of SCID mice containing a human lung tumor xenograft before and after injection of the tumor with chitinase as above. In FIG. 3a, the tumor before chitinase treatment is indicated by the arrow. FIG. 3b shows the tumor in the same mouse 10 days after a single injection of 5 units of chitinase. The tumor was substantially reduced in size after the chitinase treatment.

FIG. 3c and d show hemotoxylin and eosin (H&E) staining of sections of human lung tumor xenograft tissue from SCID mice before (FIG. 3c) and 12 days after (FIG. 3d) a single intratumoral injection of 5 units of chitinase.

### Example 4

### Survival of SCID Mice Carrying Human Bladder Cancer Xenografts After Chitinase Injection

Poorly differentiated carcinoma from human bladder was obtained from patients as a biopsy sample and a 0.1 cm³ piece of the biopsy was implanted into SCID mice. After the tumors grew to a size of between 0.3-0.6 cm³, 5 units of chitinase was injected into the tumor (day 1). Subsequently, the size of the tumors was measured daily. The results showed that the bladder tumors were as effectively eliminated from the animals by the chitinase as was the human colon cancer as described in Example 2.

### Example 5

### Survival of SCID Mice Carrying Human Malignant Melanoma Xenografts After Chitinase Injection

Malignant melanoma was obtained from patients as a biopsy sample and a 0.1 cm³ piece of the biopsy was implanted into SCID mice. After the tumors grew to a size of between 0.3-0.6 cm³, 5 units of chitinase was injected into the tumor (day 1). Subsequently, the size of the tumors was measured daily. The results showed that the melanomas were as effectively eliminated from the mouse by chitinase as was the human colon cancer as described in Example 2.

FIG. 3e and f show hemotoxylin and eosin (H&E) staining of sections of human melanoma xenograft tissue from SCID mice before (FIG. 3e) and 12 days after (FIG. 3f) a single intratumoral injection of 5 units of chitinase.

### Example 6

### Survival of Human Fibrosarcoma Xenografts in SCID Mice After Chitinase Injection

A fibrosarcoma was obtained from patients as a biopsy sample and a 0.1 cm³ piece of the biopsy was implanted into SCID mice. After the tumors grew to a size of between 0.3-0.6 cm³, 5 units of chitinase was injected into the tumor (day 1). Subsequently, the size of the tumors was measured daily. The results showed that the fibrosarcomas were as effectively eliminated from the mouse by chitinase as was the human colon cancer as described in Example 2.

### Example 7

### Survival of Human Ovarian Cancer Xenografts in SCID Mice After Chitinase Injection

Adenocarcinoma from ovaries was obtained from patients as a biopsy sample and a 0.1 cm³ piece of the biopsy was implanted into SCID mice. After the tumors grew to a size of between 0.3-0.6 cm³, 5 units of chitinase was injected into the tumor (day 1). Subsequently, the size of the tumors was measured daily. The results showed that the ovarian tumors were as effectively eliminated from the mouse by chitinase as was the human colon cancer as described in Example 2.

### Example 8

### Survival of Human Breast Cancer Xenografts in SCID Mice After Chitinase Injection

Ductal carcinoma from breast was obtained from patients as a biopsy sample and a 0.1 cm³ piece of the biopsy was implanted into SCID mice. After the tumors grew to a size of between 0.3-0.6 cm³, 5 units of chitinase was injected into the tumor (day 1). Subsequently, the size of the tumors was measured daily. The results showed that the breast tumors were as effectively eliminated from the mouse by chitinase as was the human colon cancer as described in Example 2. Similar results were achieved one million MCF-7 cells (a human breast cancer cell line) were injected into the mice and tumors resulting from growth of the cells were treated with chitinase.

### Example 9

### Survival of Merkel Cell Cancer Xenografts in SCID Mice After Chitinase Injection

Merkel cell cancer (neuroendocrine skin cancer) was obtained from patients as a biopsy sample and a 0.1 cm³ piece of the biopsy was implanted into SCID mice. After the tumors grew to a size of between 0.3-0.6 cm³, 5 units of chitinase was injected into the tumor (day 1). Subsequently, the size of the tumors was measured daily. The results showed that the Merkel cell tumors were eliminated from the mouse by chitinase as was the human colon cancer as described in Example 2.

### Example 10

### Survival of Human Prostate Cancer Xenografts in SCID Mice After Chitinase Injection

Moderately differentiated adenocarcinoma from prostate was obtained from patients as a biopsy sample and a 0.1 cm³ piece of the biopsy was implanted into SCID mice. After the tumors grew to a size of between 0.3-0.6 cm³, 5 units of chitinase was injected into the tumor (day 1). Subsequently, the size of the tumors was measured daily. The results showed that the prostate tumors were eliminated from the mouse by chitinase over a period of 7 days as shown in FIG. 2.

### Example 11

### Survival of Human Lymphoma Xenografts in SCID Mice After Chitinase Injection

Large cell lymphoma was obtained from patients as a biopsy sample and a 0.1 cm³ piece of the biopsy was implanted into SCID mice. After the tumors grew to a size of between 0.3-0.6 cm³, 5 units of chitinase was injected into the tumor (day 1). Subsequently, the size of the tumors was measured daily. The results showed that the lymphoma was reduced in size over a period of 7 days, as shown in FIG. 2.

### Example 12

### Survival of Human LNCaP Prostate Cancer Cells in SCID Mice After Chitinase Injection

One million LNCaP prostate cancer cells were injected into SCID mice. After the tumors grew to a size of between 0.3-0.6 cm³, 5 units of Streptomyces chitinase was injected intratumorally, intraperitoneally and intravenously, in separate mice. Tumor necrosis was visible within hours after injection and analysis of the tumors by histological staining showed extensive cellular damage and morphological changes compared to control tumors, treated with injection of saline. There was complete tumor regression in animals given chitinase. The treated animals survived without tumor recurrence for more than one year. All untreated animals died within 3 months. Similar tumor regression data were also obtained in murine models carrying xenografts of human breast cancer and human colon cancer.

Additional studies showed that tumors could be more rapidly eliminated from the mice by an additional administration of chitinase give after the first administration. In addition, there was no indication of development of resistance of the tumors to the enzyme.

### Example 13

### Growth of Murine Sarcoma Tumors in C57BL/6 Mice After Chitinase Injection

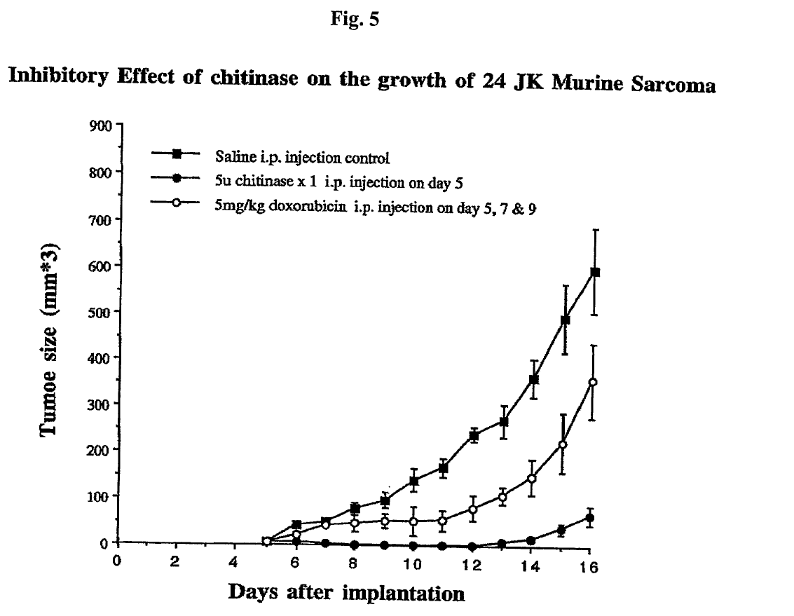

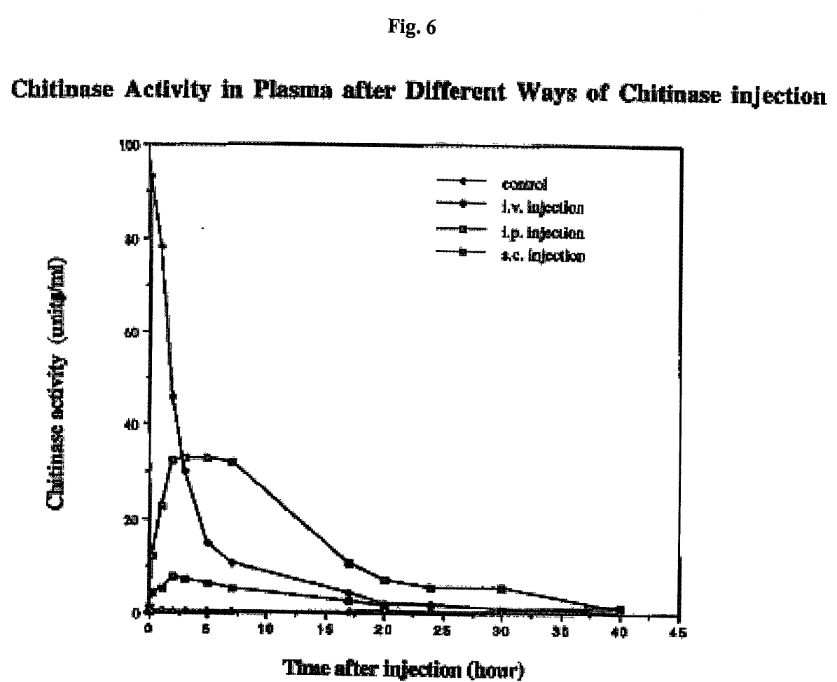

A syngeneic tumor model, C57BL/6 mice carrying implanted 24 JK murine sarcoma cells, was used in these studies. The cells were injected subcutaneously into the mouse flank. Five days later, 5 units of chitinase was injected intraperitoneally (IP) into the mouse. Into another mouse was injected 5 mg doxorubicin per kilogram of mouse weight IP on days 5, 7 and 9. Into a third mouse was injected saline on day 5 (i.e., negative control). The data in FIG. 5 show that a single injection of the chitinase inhibited detectable growth of the 24 JK sarcoma tumor cells until after day 12. In contrast, the standard chemotherapy agent, doxorubicin, was generally ineffective and mice evaluated eventually died from drug toxicity or progressive tumor growth. Mice treated with 3 intraperitoneal injections of 5 mg doxorubicin/kg of mouse body weight (i.e., at the maximum tolerated dose) showed growth attenuation followed by a resumption of progressive growth. Tumor-bearing animals treated with placebo (saline) showed progressive tumor growth.

Additional studies showed that complete tumor regression of established 24 JK murine sarcoma tumors was achieved by intraperitoneal injection of 5 Units of chitinase on day 5 and day 6, or a single intraperitoneal injection of 10 Units of chitinase on day 5.

Additional studies showed that anti-tumor activity of chitinase was dose dependent and increased with increasing dosages of the enzyme. Heat-inactivated chitinase was ineffective at tumor reduction.

### Example 12

### Half-Life of Chitinase Given to Mice Using Different Routes of Administration

Thirty-five mg of chitinase (Sigma C-6139) was dissolved in 1.5 ml of PBS. One-half ml of the chitinase solution was injected into each of 3 mice. One mouse was injected intraperitoneally (IP), one was injected intravenously (IV), and one mouse was injected subcutaneously (SC). A fourth mouse was injected with saline as a control. At various times after injection, 15-20 μl of blood was taken from the tail vein of each mouse and immediately mixed with a sodium-EDTA solution (pH 7.4). The cells were centrifuged from the blood and 5 μl of plasma was used in a chitinase assay. In the chitinase assay, 4-methylumbelliferyl-β-D-N, N′, N″-triacetyl-chitotriose (4-mu-chitotrioside) was used as the substrate for chitinase. The plasma sample, in 100 μl of saline, was mixed with 100 μl of 22 μM 4-mu-chitotrioside solution in 0.2 M citric acid buffer (pH 5.2) and incubated in a 37° C. water bath for 15 min. At the end of the incubation, 3.0 ml of 0.3 M glycine buffer (pH 10.6) was added to the mixture to stop the reaction. The fluorescence of the solution at 460 nm was then measured after excitation at 355 nm.

The results of this study are shown in FIG. 6. The data show that very high levels of chitinase were found in the blood immediately after (within 1 min.) IV injection of chitinase. These levels rapidly decreased over a period of 5 min. and then more gradually tapered off until 20 min., at which time, little chitinase was left in the blood. After IP injection, levels of chitinase in the blood rapidly increased until maximum levels (less than half of those found in IV injection) were reached at about 2-3 min. after injection. These levels remained until 7-8 min. after injection, then decreased rapidly until 20 min., then tapered off more slowly until 40 min. After SC injection, chitinase levels in the blood maximally reached only a third of the blood levels found after IP injection. These levels were reached 2-3 min. after injection and then tapered off slowly until 40 min. post-chitinase injection.

### Example 13

### A Hexosaminidase-Ligand Conjugate with Folate as the Targeting Ligand

A folate-chitinase conjugate is synthesized. First, N-hydroxysuccinimidyl-folate (NHS-folate) is synthesized by reacting folic acid with N-hydroxysuccinimide (NHS) in the presence of dicyclohexylcarbodiimide (DCC) in DMSO. Next, folic acid is covalently coupled to chitinase by reacting NHS-folate with the ε-amino groups on the lysine side chains of the protein. One to three folates are coupled to each molecule of chitinase. Then, the specific activity of the folate conjugate is determined by chitinase enzyme assay to measure any losses in enzyme activity due to folate conjugation. In vitro cellular binding studies are then carried out to determine the affinity of folate conjugated chitinase for tumor cells.

Preferably, a biodistribution study is performed to study the in vivo tumor localizing properties of folate-conjugated chitin derivatives. Chitinase expression in tumor tissues is evaluated by immunohistochemical staining and compared to tumors from mice treated with non-folate-conjugated chitinase derivatives. This determines whether folate targeting of chitinase or chitinase elicitors leads to tumor-specific increases in chitinase levels within the tumor. An anti-tumor efficacy study is then be carried out as described above to access the therapeutic potential of these folate conjugates.

### Example 14

### Stimulation of Chitinase Production in Macrophages by Chitin Derivatives

To evaluate the anti-tumor activity of chitin derivatives as chitinase elicitors, mice are treated with varying doses of these derivatives. Serum chitinase activity is measured to quantify the stimulatory effect of each compound. The derivative with the greatest stimulatory effect is then tested in mice bearing prostate cancer xenograft for anti-tumor activity, as described above. Furthermore, native chitinase production can be stimulated by chitin derivatives, which circumvents the need to administer a protein drug with poor bioavailabilty.

While the above description contains many specificities, these should not be construed as limitations on the scope of the invention, but rather as exemplification of preferred embodiments. Numerous other variations of the present invention are possible, and is not intended herein to mention all of the possible equivalent forms or ramifications of this invention. Various changes may be made to the present invention without departing from the scope of the invention.

We claim:

1. A hexosaminidase-ligand conjugate for treating cancer, comprising:
(a) a hexosaminidase, and
(b) a cancer cell targeting ligand covalently bound to the hexosaminidase.

2. The conjugate of claim 1, further comprising a linker molecule covalently attached to both the hexosaminidase and the targeting ligand.

3. The conjugate of claim 2 wherein said linker molecule is polyethylene glycol (PEG).

4. The conjugate of claim 1, wherein said targeting ligand is selected from the group consisting of a monoclonal antibody immunospecific to a tumor cell or cancer cell antigen, an antibody fragment immunospecific to a tumor cell or cancer cell antigen, epidermal growth factor (EGF), fibroblast growth factor (FGF), interleukin-2 (IL-2), transferrin, somatostatin, and folic acid.

5. The conjugate of claim 1, wherein said targeting ligand is folic acid.

6. The conjugate of claim 1, wherein said hexosaminidase is an endoglycosidase or an exoglycosidase.

7. The conjugate of claim 1, wherein said hexosaminidase is selected from the group consisting of chitinase, chitosanase, and N-acetyl-hexosaminidase.

8. The conjugate of claim 7, wherein said chitinase is N-acetyl-glucosaminohydrolase.

9. The conjugate of claim 1, wherein said hexosaminidase is chitinase.

10. The conjugate of claim 1, wherein said hexosaminidase is derived from bacteria, yeast, fungus, plants, or mammalian sources.

11. The conjugate of claim 1 wherein the targeting ligand binds or associates with a cancer cell selected from the group colon cancer cell, lung cancer cell, bladder cancer cell, lymphoma cell, melanoma cell, fibrosarcoma cell, Merkel cell, ovarian cancer cell, breast cancer cell, prostate cancer cell and oral carcinoma cell.

12. The conjugate of claim 1 wherein the hexosaminidase is chitinase and the targeting ligand is folic acid.

13. A pharmaceutical composition comprising the conjugate of claim 1, and one or more additives selected from the group consisting of salts, buffering agents, preservatives, adjuvants, vehicles, pharmaceutically-acceptable carriers, and other therapeutic agents.

14. A hexosaminidase-ligand conjugate for treating cancer, comprising:
(a) a hexosaminidase,
(b) a cancer cell targeting ligand which is a protein,
wherein said conjugate is a fusion protein.

15. A method for treating cancer in a subject, comprising administering a pharmaceutical composition comprising a hexosaminidase to a subject.

16. The method of claim 15 wherein the method of administration is selected from the group consisting of intravenously, intraperitoneally, intramuscularly, intracerebrally, and directly into said cancer.

17. The method of claim 15 wherein the cancer is selected from the group colon cancer, lung cancer, bladder cancer, lymphoma, melanoma, fibrosarcoma, Merkel cell, ovarian, breast, prostate cancer and oral carcinoma.

18. A method for treating cancer in a subject, comprising administering a pharmaceutical composition comprising a hexosaminidase-ligand conjugate to a subject.

19. The method of claim 15 wherein the method of administration is selected from the group consisting of intravenously, intraperitoneally, intramuscularly, intracerebrally, and intratumorally.

20. The method of claim 13 or 15, further comprising the step of combining said method for treating cancer with chemotherapy, surgery, radiotherapy, photodynamic therapy, gene thereapy, antisense therapy, enzyme prodrug therapy, immunotherapy, fusion toxin therapy, antiangiogenic therapy, or a combination thereof.

21. The method of claim 18 wherein the cancer is selected from the group colon cancer, lung cancer, bladder cancer, lymphoma, melanoma, fibrosarcoma, Merkel cell, ovarian, breast, prostate and oral carcinoma.

22. A method for treating cancerous tumors in a patient, comprising: administering a chitin derivative to the patient to stimulate production of chitotriosidase by the patient's macrophages or monocytes.

23. The method of claim 22, wherein said chitin derivative is chitosan or a derivative thereof.

24. A method for treating a subject with cancer, comprising: administering an expression vector comprising a polynucleotide encoding chitinase to the subject wherein expression of said polynucleotide results in production of chitinase in the subject.
